(19) United States
(12) Patent Application Publication
(10) Pub. No.: US 2020/0178762 A1
(43) Pub. Date: Jun. 11, 2020

(54) INSERTION DEVICE

(71) Applicant: OLYMPUS CORPORATION, Tokyo (JP)

(72) Inventor: Kohei ARAKI, Tokyo (JP)

(73) Assignee: OLYMPUS CORPORATION, Tokyo (JP)

(21) Appl. No.: 16/792,993

(22) Filed: Feb. 18, 2020

Related U.S. Application Data
(63) Continuation of application No. PCT/JP2018/028482, filed on Jul. 30, 2018.

(30) Foreign Application Priority Data
Aug. 23, 2017 (JP) 2017-160610

(51) Int. Cl. A61B 1/005; A61B 1/008

(52) U.S. Cl. CPC A61B 1/0055; A61B 1/00045; A61B 1/008

(57) ABSTRACT

An endoscope includes an active bending portion that is configured to be bent in a first direction according to a bending operation of an operator, a passive bending portion that is provided on a proximal end side of the active bending portion and is passively bent by receiving an external force without being bent according to the bending operation of the operator, and a flexible tube. The passive bending portion includes a first area including two coupling portions that couple two adjacent bending pieces rotatably around two rotation axes crossing at a first angle around a central axis of an insertion portion with respect to a second direction orthogonal to the first direction and a second area including two coupling portions that couple another two adjacent bending pieces rotatably around two second rotation axes crossing at a second angle larger than the first angle with respect to the second direction.

### CROSS REFERENCE TO RELATED APPLICATION

This application is a continuation application of PCT/JP2018/028482 filed on Jul. 30, 2018 and claims benefit of Japanese Application No. 2017-160610 filed in Japan on Aug. 23, 2017, the entire contents of which are incorporated herein by this reference.

### BACKGROUND OF THE INVENTION

### 1. Field of the Invention

The present invention relates to an insertion device, and more particularly, to an insertion device including an insertion portion provided with a bending portion on a distal end side.

### 2. Description of the Related Art

Conventionally, insertion devices such as endoscopes have been widely used. In a case of an endoscope, an insertion portion is inserted into a subject, an image in the subject is obtained, an endoscopic image is displayed on a display apparatus, and the like to thereby perform an inspection in the subject. A bending portion that can be freely bent by an operation of an operator such as a doctor, that is, an active bending portion is provided on a distal end side of the insertion portion of the insertion device.

Further, as disclosed in Japanese Patent Application Laid-Open Publication No. 2006-218231 and International Publication No. WO2011/136115, there is also proposed an insertion portion including a flexible passive bending portion that cannot be bent by a bending operation of the operator but can be passively bent when receiving an external force on a proximal end side in an insertion direction from the active bending portion in the insertion portion.

In the proposed insertion portion, there is prevented an occurrence of a phenomenon, which occurs, for example, when the insertion portion passes through intestines, such that a curvature radius of the passive bending portion is made larger than a curvature radius of the active bending portion, and thereby when the insertion portion is pressed into the subject, an intestine wall is pressed by a portion in which the insertion portion is bent, what is called a stick phenomenon. In other words, a curvature radius of the passive bending portion is made larger than a curvature radius of the active bending portion and a bending angle of a bending part including the active bending portion and the passive bending portion is made gentle. Thereby, there is prevented an occurrence of a phenomenon such that as the insertion portion is pressed, the bending portion knocks up a flexed portion of an intestines, which results from an extremely large bending angle of the bending portion.

In order to insert the bending portion in conformity to a flexed direction of the flexed portion such as an intestine canal, when the operator presses the insertion portion, it is necessary to bend the passive bending portion in conformity to the flexed direction.

### SUMMARY OF THE INVENTION

An insertion device according to one aspect of the present invention includes an insertion portion that is inserted into a subject from a distal end side in a longitudinal axis direction, wherein the insertion portion includes a distal end portion that is provided at a distal end of the insertion portion, a first bending portion that is provided on a proximal end side of the distal end portion and is configured to be bent in a first direction according to a bending operation of an operator, a second bending portion that is provided on a proximal end side of the first bending portion and is passively bent by receiving an external force without being bent by the bending operation of the operator, and a flexible tube that is provided on a proximal end side of the second bending portion and has flexibility, the second bending portion includes a first area including a first coupling portion that couples two adjacent bending pieces rotatably around a first rotation axis crossing in a positive direction at a first angle around a central axis of the insertion portion with respect to a second direction orthogonal to the first direction and a second coupling portion that couples the two adjacent bending pieces rotatably around a second rotation axis crossing in a negative direction at the first angle around the central axis with respect to the second direction, and a second area including a third coupling portion that is provided on a proximal end side from the first area and couples another two adjacent bending pieces rotatably around a third rotation axis crossing in the positive direction at a second angle around the central axis with respect to the second direction and a fourth coupling portion that couples the other two adjacent bending pieces rotatably around a fourth rotation axis crossing in the negative direction at the second angle around the central axis with respect to the second direction, and an absolute value of the second angle is larger than an absolute value of the first angle.

### BRIEF DESCRIPTION OF THE DRAWINGS

FIG. 1 is a schematic view of an endoscope 1 according to a first embodiment of the present invention;

FIG. 2 is a partial cross-sectional view of a distal end portion provided in an insertion portion 2 of the endoscope 1 according to the first embodiment of the present invention;

FIG. 3 is a cross-sectional view of an active bending portion 14 provided in the insertion portion 2 of the endoscope 1 according to the first embodiment of the present invention;

FIG. 4 is a partially sectional view of a passive bending portion 15 provided in the insertion portion 2 of the endoscope 1 according to the first embodiment of the present invention;

FIG. 5 is a cross-sectional view of the passive bending portion 15 along a V-V line shown in FIG. 4;

FIG. 6 is a cross-sectional view of the passive bending portion 15 along a VI-VI line shown in FIG. 4;

FIG. 7 is a cross-sectional view of the passive bending portion 15 along a VII-VII line shown in FIG. 4;

FIG. 8 is a perspective view of a plurality of bending pieces 41 of the bent passive bending portion 15 according to the first embodiment of the present invention;

FIG. 9 is a diagram describing a curvature radius of the passive bending portion 15 in a bent state according to the first embodiment of the present invention;

FIG. 10 is a diagram describing a bendable range in an up-down direction (UD direction) of the passive bending portion 15 according to the first embodiment of the present invention;

FIG. 11 is a diagram showing a component force applied to a rotation axis when pressing force of a large intestine is applied from an up direction to an area BA1 of the passive bending portion 15 according to the first embodiment of the present invention;

FIG. 12 is a diagram showing a component force applied to the rotation axis when the pressing force of the large intestine is applied from an up direction to an area BA2 of the passive bending portion 15 according to the first embodiment of the present invention;

FIG. 13 is a diagram showing a component force applied to the rotation axis when the pressing force of the large intestine is applied from an up direction to an area BA3 of the passive bending portion 15 according to the first embodiment of the present invention;

FIG. 14 is a diagram describing an operation for inserting the insertion portion 2 of the endoscope 1 into a flexed portion BP in a large intestine according to the first embodiment of the present invention;

FIG. 15 is a diagram describing an operation for inserting the insertion portion 2 of the endoscope 1 into the flexed portion BP in a large intestine according to the first embodiment of the present invention;

FIG. 16 is a diagram describing an operation for inserting the insertion portion 2 of the endoscope 1 into the flexed portion BP in a large intestine according to the first embodiment of the present invention;

FIG. 17 is a diagram describing an operation for inserting the insertion portion 2 of the endoscope 1 into the flexed portion BP in a large intestine according to the first embodiment of the present invention;

FIG. 18 is a front view of the plurality of bending pieces 41 showing a coupling relationship between the bending pieces 41 of a passive bending portion 15A according to a second embodiment of the present invention;

FIG. 19 is a cross-sectional view along an XIX-XIX line shown in FIG. 18;

FIG. 20 is a perspective view of the plurality of bending pieces 41 of the bent passive bending portion 15A according to the second embodiment of the present invention;

FIG. 21 is a front view of the plurality of bending pieces 41 showing a coupling relationship between the bending pieces 41 of a passive bending portion 15B according to a third embodiment of the present invention; and

FIG. 22 is a front view of the plurality of bending pieces 41 showing a coupling relationship between the bending pieces 41 of the passive bending portion 15B according to the third embodiment of the present invention.

### DETAILED DESCRIPTION OF THE PREFERRED EMBODIMENTS

Hereinafter, embodiments of the present invention will be described below with reference to the drawings. Note that it should be noticed that the drawings are schematic, and a relationship between a thickness and a width of each member, a thickness ratio among respective members and the like are different from actual ones, and it goes without saying that, among the drawings, portions that are different in mutual dimensional relationship and ratio are included.

### First Embodiment

(Configuration of the Entire Endoscope)

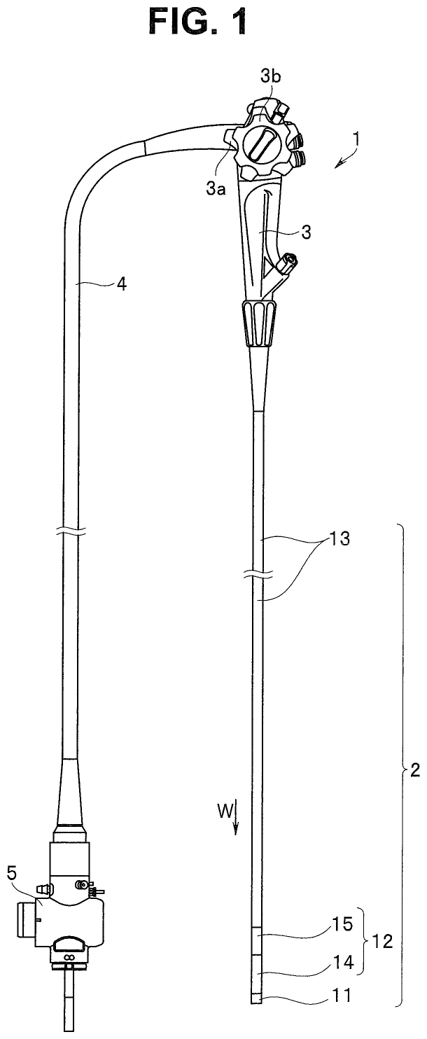

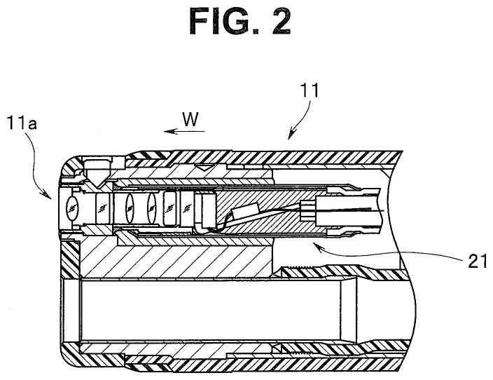

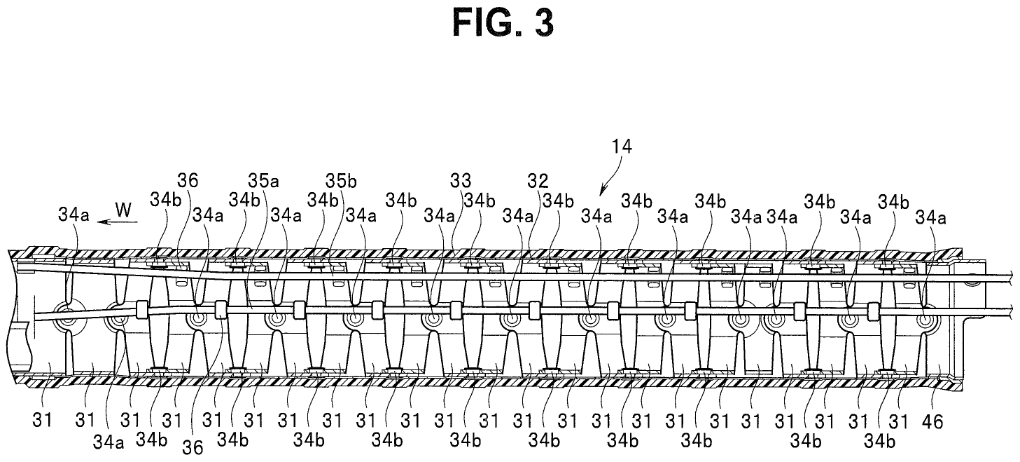

FIG. 1 is a schematic view of an endoscope 1 according to a first embodiment. FIG. 2 is a partial cross-sectional view of a distal end portion provided in an insertion portion 2 of the endoscope 1 shown in FIG. 1. FIG. 3 is a cross-sectional view of an active bending portion 14 provided in the insertion portion 2 of the endoscope 1 shown in FIG. 1.

As shown in FIG. 1, a principal part of the endoscope 1 includes the insertion portion 2 that is inserted into a subject, an operation portion 3 that is connected to a proximal end side of the insertion portion 2, a universal code 4 that is extended from the operation portion 3, and a connector 5 that is provided in an extended end of the universal code 4. Note that the endoscope 1 is electrically connected to an external apparatus such as a control apparatus or a lighting apparatus via the connector 5.

The operation portion 3 is provided with an up-down bending operation knob (hereinafter, simply referred to as a knob) 3a configured to bendingly operate the active bending portion 14 to be described later and a left-right bending operation knob (hereinafter, simply referred to as a knob) 3b.

As shown in FIG. 2, an image pickup unit 21 that observes the inside of the subject, an illumination unit (not shown) that illuminates the inside of the subject, and the like are provided in a distal end portion 11 provided in a distal end of the insertion portion 2. The image pickup unit 21 is provided on the rear side of an observation window 11a of the distal end portion 11.

In other words, the image pickup unit 21 functioning as an image acquisition apparatus that picks up an image of the subject is provided on a distal end side in a longitudinal axis direction from the active bending portion 14.

The insertion portion 2 includes the distal end portion 11, a bending portion 12, and a flexible tube 13 in order from the distal end, and is formed in an elongated shape along an insertion direction W of the insertion portion 2. The insertion portion 2 is configured so as to be inserted into the subject from the distal end side in the longitudinal axis direction of the insertion portion 2. In other words, the endoscope 1 configures an insertion device having the insertion portion 2 that is inserted into the subject from the distal end side in the longitudinal axis direction.

The bending portion 12 includes the active bending portion 14 that is a first bending portion and a passive bending portion 15 that is a second bending portion in order from the distal end.

(Configuration of Flexible Tube)

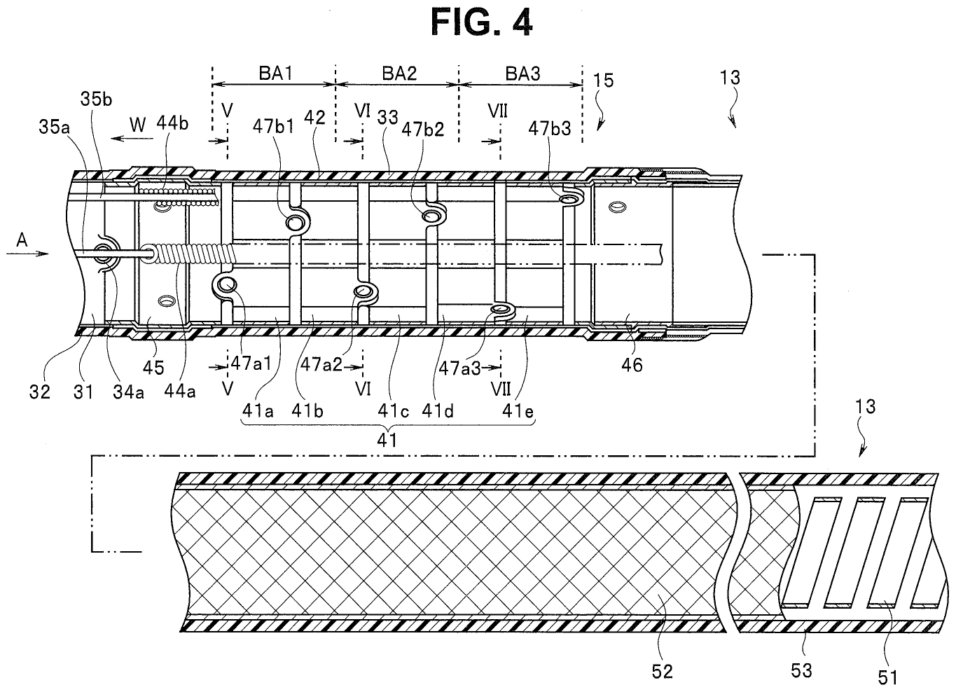

As shown in FIG. 4 to be described later, the flexible tube 13 has a hollow shape. As will be described later, the flexible tube 13 includes a spiral tube 51 that is formed by spirally winding a strip-shaped element wire such as a thin plate member, a net-like tube 52 that is provided on an outer peripheral side (on an outer peripheral surface) of the spiral tube 51 and is formed into a tubular shape by braiding fibers such as metal or resin, and an outer skin 53 that is provided on an outer peripheral side (on an outer peripheral surface) of the net-like tube 52 and has flexibility. The flexible tube 13 is provided on the proximal end side of the passive bending portion 15 and has flexibility.

(Configuration of Active Bending Portion)

The active bending portion 14 that is the first bending portion is bendable in an up-down direction as a first direction, a left-right direction as a second direction, and further by 360° in a direction combining four directions of up, down, left, and right by pulling or slackening bending wires 35a to 35d (bending wires 35c and 35d are not shown in FIG. 3) to be described later inserted into the insertion portion 2 by the bending operation (here, operations of the knob 3a and the knob 3b) of the operator. In other words, according to the bending operation of the operator, when the insertion portion 2 is viewed from the distal end side, the active bending portion 14 can be bent not only in the up-down direction but also in the left-right direction.

Specifically, as shown in FIG. 3, a principal part of the active bending portion 14 includes a plurality of bending pieces 31, a blade 32, which is a net-like tube, that covers an outer periphery of the plurality of bending pieces 31, and an outer skin resin 33 that covers an outer periphery of the blade 32. Each bending piece 31 has an annular shape and is made of metal such as stainless steel.

Note that, here, the up-down direction is a direction substantially parallel to an up-down direction of a screen when an endoscopic image obtained by picking up an image by the image pickup unit 21 is displayed on the screen of a display apparatus. Further, the left-right direction is a direction substantially parallel to a left-right direction of the screen when the obtained endoscopic image is displayed on the screen of the display apparatus.

As shown in FIG. 3, the plurality of bending pieces 31 are coupled so that each bending piece 31 is rotatable around a predetermined rotation axis along the insertion direction W (distal end direction of the insertion portion 2). In other words, between two adjacent bending pieces 31 in the insertion direction W, the plurality of bending pieces 31 are coupled rotatably by rivets 34a and 34b as an example, here, around a plurality of rotation axes constituting rotation axes positioned differently from each other by 90° in a circumferential direction J of the bending pieces 31.

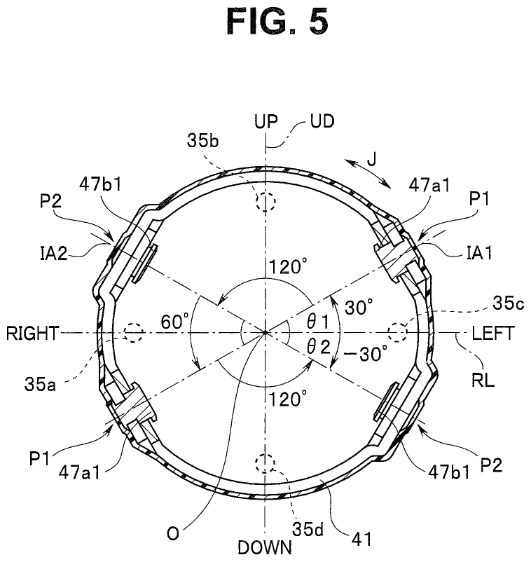

More specifically, the adjacent bending pieces 31 in the insertion direction W are coupled rotatably in the up-down direction by two opposing rivets 34a (only one rivet is shown in FIG. 3). At the same time, the adjacent bending pieces 31 are coupled rotatably in the left-right direction by two opposing rivets 34b in a position different from the rivet 34a by 90° in the circumferential direction J. A first rotation axis RL (defined in FIG. 5) is constituted by the two rivets 34a, and a second rotation axis UD (defined in FIG. 5) is constituted by the two rivets 34b.

Note that, as shown in FIG. 3, the adjacent bending pieces 31 are alternately coupled by the rivet 34a and the rivet 34b between the bending pieces 31, for example, so that in a case in which a first bending piece 31 and a second bending piece 31 are coupled by the rivet 34a, the second bending piece 31 and a third bending piece 31 are coupled by the rivet 34b, further the third bending piece 31 and a fourth bending piece 31 are coupled by the rivet 34a, and the like.

As a result, the active bending portion 14 has a configuration that is bendable by 360° in up, down, left, and right directions and in a direction combining four directions of the up, down, left, and right directions. In other words, the active bending portion 14 can be bent in a plurality of directions by the first rotation axis RL and the second rotation axis UD shown in FIG. 5.

Note that, as shown in FIG. 3, four bending wires 35a to 35d (only the bending wires 35a and 35b are shown in FIG. 3) positioned differently from each other by 90° are inserted into the active bending portion 14 in the circumferential direction J of the bending pieces 31. Two bending wires 35a and 35c are arranged in the same positions as the positions of the two rivets 34a in the circumferential direction J along the central axis of the insertion portion 2. Two bending wires 35b and 35d are arranged in the same positions as the positions of the two rivets 34b in the circumferential direction J along the central axis of the insertion portion 2.

Further, the four bending wires 35a to 35d are supported by wire receivers 36 provided in respective bending pieces 31 in the active bending portion 14. Further, distal ends of respective wires 35a to 35d are connected to the bending piece 31 positioned on the most distal end side in the insertion direction W among the plurality of bending pieces 31. As a result, as the bending wires 35a to 35d are pulled and slackened, the respective bending pieces 31 rotate around the rotation axis of either the rivet 34a or the rivet 34b, and the active bending portion 14 is bent.

As described above, the active bending portion 14 is provided on the proximal end side of the distal end portion 11 and constitutes the first bending portion that is bent in the up, down, left, and right directions according to the bending operation of the operator. The active bending portion 14 that is the first bending portion includes the plurality of serially coupled bending pieces 31. Here, the up-down direction in the bending is substantially parallel to an up-down direction of the image obtained by the image acquisition apparatus and displayed on the screen of the display apparatus. Further, the left-right direction is substantially parallel to a left-right direction of the image.

(Configuration of Passive Bending Portion)

The passive bending portion 15 cannot be bent according to the bending operation of the operator. However, when receiving an external force, the passive bending portion 15 is passively bendable in four up, down, left, and right directions or in the direction combining the four up, down, left, and right directions. In other words, the passive bending portion 15 constitutes the second bending portion that is provided on a proximal end side of the active bending portion 14, is not actively bent by the bending wires or other bending operation means according to the bending operation of the operator, and is passively bent by receiving an external force.

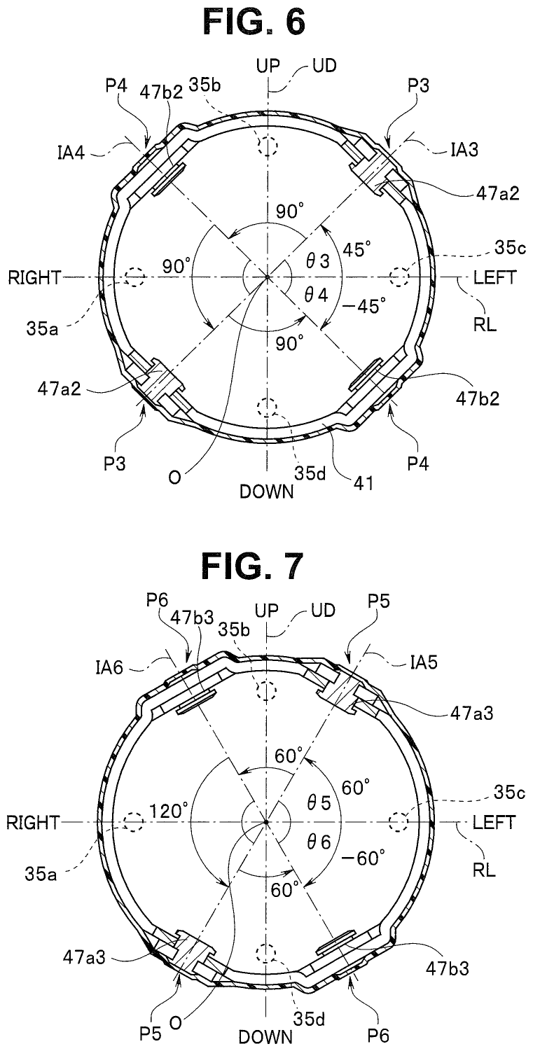

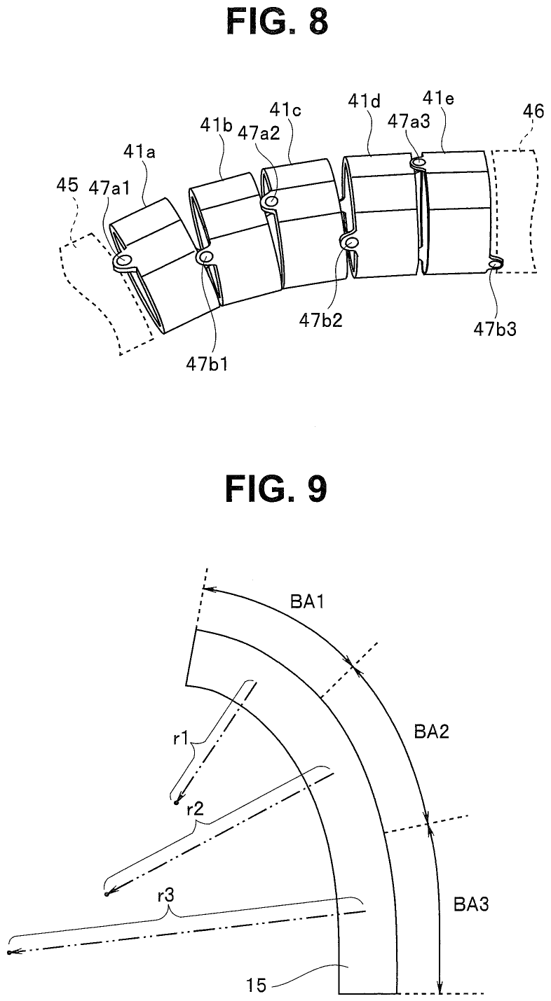

FIG. 4 is a partial cross-sectional view of the passive bending portion 15 provided in the insertion portion 2 of the endoscope 1 shown in FIG. 1. FIG. 5 is a cross-sectional view of the passive bending portion 15 along a V-V line shown in FIG. 4. FIG. 6 is a cross-sectional view of the passive bending portion 15 along a VI-VI line shown in FIG. 4. FIG. 7 is a cross-sectional view of the passive bending portion 15 along a VII-VII line shown in FIG. 4. FIG. 5 to FIG. 7 are diagrams viewed from a direction of an arrow A shown in FIG. 4. FIG. 8 is a perspective view of a plurality of bending pieces of the bent passive bending portion 15. FIG. 9 is a diagram describing a curvature radius of the passive bending portion 15 in the bent state.

As shown in FIG. 4, a principal part of the passive bending portion 15 includes a plurality of bending pieces 41a to 41e (hereinafter, referred to as the bending piece 41 when indicating a plurality of bending pieces or arbitrary one bending piece), a blade 42 that covers an outer periphery of the plurality of bending pieces 41, and the outer skin resin 33 that covers an outer periphery of the blade 42. The each bending piece 41 has an annular shape and is made of metal such as stainless steel. In other words, the passive bending portion 15 includes the plurality of annular bending pieces 41 coupled in series.

The four bending wires 35a to 35d described above are inserted into the plurality of bending pieces 41 of the passive bending portion 15. Note, however, that respective bending wires 35a to 35d are not connected to any one of the plurality of bending pieces 41 such as the bending piece 41a positioned on the most distal end side in the insertion direction W. Outer circumferences of the four bending wires 35a to 35d are covered with known coil pipes 44a to 44d (the coil pipes 44c and 44d are not shown in FIG. 4). Distal ends of the coil pipes 44a to 44d are fixed to a pipe sleeve 45 to be described later by welding or the like.

The passive bending portion 15 includes the plurality of bending pieces 41. The plurality of bending pieces 41 are coupled along the insertion direction W so that the passive bending portion 15 can be bent. In the plurality of bending pieces 41, both of the two adjacent bending pieces 41 in the insertion direction W are coupled by two rivets provided in predetermined positions in the circumferential direction J of respective bending pieces 41.

The passive bending portion 15 has a plurality of areas, here, three areas BA1, BA2, and BA3 from the active bending portion 14 toward the flexible tube 13, namely, from the distal end side toward the proximal end side.

Note that, here, the passive bending portion 15 has the three areas BA1, BA2, and BA3, and further may have two areas or four areas or more.

More specifically, as shown in FIG. 5, when the passive bending portion 15 is viewed from the distal end side of the insertion portion 2, an axis in the left-right direction passing through the central axis O of the insertion portion 2 shows the first rotation axis RL described above of the active bending portion 14 for bending the active bending portion 14 in the up-down direction by pulling and slackening the two bending wires 35b and 35d.

Similarly, when the passive bending portion 15 is viewed from the distal end side of the insertion portion 2, an axis in the up-down direction passing through the central axis O of the insertion portion 2 shows the second rotation axis UD described above of the active bending portion 14 for bending the active bending portion 14 in the left-right direction by pulling and slackening the two bending wires 35a and 35c.

The bending piece 31 at the most proximal end of the active bending portion 14 and the bending piece 41a at the most distal end of the passive bending portion 15 are connected via the pipe sleeve 45. A bending piece 41e at the most proximal end of the passive bending portion 15 and the distal end portion of the flexible tube 13 are connected via a pipe sleeve 46.

As shown in FIG. 4, before the outer skin resin 33 is covered, the active bending portion 14 and the passive bending portion 15 are connected via the pipe sleeve 46 in a state in which the outer periphery of the respective bending pieces 31 and 41 is covered with the blades 32 and 42.

As shown in FIG. 5, in the area BA1 on the distal end side of the passive bending portion 15, the pipe sleeve 45 and the bending piece 41a adjacent to each other in the insertion direction W are connected by two rivets 47a1 in two positions P1 on a third rotation axis IA1 inclined by a predetermined first angle θ1 (here, 30°) with respect to the first rotation axis RL in a counterclockwise direction around the central axis O of the insertion portion 2. Two bending pieces 41a and 41b are connected by two rivets 47b1 in two positions P2 on a fourth rotation axis IA2 inclined by a predetermined second angle θ2 (here, −30°) with respect to the first rotation axis RL in a counterclockwise direction around the central axis O of the insertion portion 2.

Therefore, the bending piece 41a at the most distal end is rotatable around the third rotation axis IA1. The bending piece 41b is rotatable around the fourth rotation axis IA2.

In other words, when the passive bending portion 15 is viewed from the distal end side of the insertion portion 2, the third rotation axis IA1 is inclined by the predetermined first angle θ1 (here, 30°) with respect to the first rotation axis RL. The fourth rotation axis IA2 is inclined by the predetermined second angle θ2 (here, −30°) with respect to the first rotation axis RL.

As shown in FIG. 6, in the area BA2 on the distal end side of the passive bending portion 15, two adjacent bending pieces 41b and 41c in the insertion direction W are connected by two rivets 47a2 in two positions P3 on a fifth rotation axis IA3 inclined by a predetermined third angle θ3 (here, 45°) with respect to the first rotation axis RL in a counterclockwise direction about the central axis O of the insertion portion 2. Two bending pieces 41c and 41d are connected by two rivets 47b2 in two positions P4 on a sixth rotation axis IA4 inclined by a predetermined fourth angle θ4 (here, −45°) with respect to the first rotation axis RL in a counterclockwise direction about the central axis O of the insertion portion 2.

Therefore, the bending piece 41c is rotatable around the fifth rotation axis IA3. The bending piece 41d is rotatable around the sixth rotation axis IA4.

In other words, when the passive bending portion 15 is viewed from the distal end side of the insertion portion 2, the fifth rotation axis IA3 is inclined by the predetermined third angle θ3 (here, 45°) with respect to the first rotation axis RL. The sixth rotation axis IA4 is inclined by the predetermined fourth angle θ4 (here, −45°) with respect to the first rotation axis RL.

As shown in FIG. 7, in the area BA3 on the distal end side of the passive bending portion 15, two adjacent bending pieces 41d and 41e in the insertion direction W are connected by two rivets 47a3 in two positions P5 on a seventh rotation axis IA5 inclined by a predetermined fifth angle θ5 (here, 60°) with respect to the first rotation axis RL in a counterclockwise direction about the central axis O of the insertion portion 2. The bending piece 41e and the pipe sleeve 46 are connected by two rivets 47b3 in two positions P6 on an eighth rotation axis IA6 inclined by a predetermined sixth angle θ6 (here, −60°) with respect to the first rotation axis RL in a counterclockwise direction about the central axis O of the insertion portion 2.

Therefore, the bending piece 41e is rotatable around the seventh rotation axis IA5. The bending piece 41e is rotatable around the eighth rotation axis IA6.

In other words, the seventh rotation axis IA5 is inclined by the predetermined fifth angle θ5 (here, 60°) with respect to the first rotation axis RL when the passive bending portion 15 is viewed from the distal end side of the insertion portion 2. The eighth rotation axis IA6 is inclined by the predetermined sixth angle θ6 (here, −60°) with respect to the first rotation axis RL.

The plurality of bending pieces 41 of the passive bending portion 15 are coupled while having the above-described coupling relationship. Here, two bending pieces 41 are included in the respective areas BA1, BA2, and BA3, and further three bending pieces 41 or more may be included.

In the area BA1, the each bending piece 41 is rotatable around the third rotation axis IA1 or the fourth rotation axis IA2. In the area BA2, the each bending piece 41 is rotatable around the fifth rotation axis IA3 or the sixth rotation axis IA4. In the area BA3, the each bending piece 41 is rotatable around the seventh rotation axis IA5 or the eighth rotation axis IA6.

Note that, in the plurality of bending pieces 41, a bending piece on the proximal end side of two first bending pieces 41 and a bending piece on the distal end side of two second bending pieces 41 are the same bending piece. As shown in FIG. 4, for example, according to the present embodiment, the bending piece on the proximal end side of the area BA1 and the bending piece on the distal end side of the area BA2 are the same bending piece. Further, the bending piece on the proximal end side of the area BA2 and the bending piece on the distal end side of the area BA3 are the same bending piece.

Therefore, as shown in FIG. 8, the passive bending portion 15 in which the plurality of bending pieces 41 are coupled to each other is bendable when an external force is received from a linear state along the central axis O. Therefore, the passive bending portion 15 is bendable in the up, down, left, and right directions and in the direction combining the four directions of the up, down, left, and right directions, namely, by 360° around the central axis O.

When the passive bending portion 15 is bent in the up, down, left, and right directions, the each bending piece 41 rotates around axes of all rivets 47a1, 47a2, 47a3, 47b1, 47b2, and 47b3. A part of each position of respective rivets 47a1, 47a2, 47a3, 47b1, 47b2, and 47b3 of the plurality of bending pieces 41 constitutes a coupling portion that couples two adjacent bending pieces rotatably around each rotation axis. Further, when the passive bending portion 15 is bent in an oblique direction other than the up, down, left, and right directions, the each bending piece 41 rotates around an axis of either one of the adjacent rivets.

Further, in the passive bending portion 15, as shown in FIG. 5 to FIG. 7, the rotation axes of the respective bending pieces 41 within the areas BA1, BA2, and BA3 respectively are inclined by a predetermined angle with respect to the RL direction when the insertion portion 2 is viewed from the distal end side, and therefore curvature radii differ in the UD direction.

More specifically, as shown in FIG. 9, a curvature radius r1 in the area BA1 on the distal end side of the passive bending portion 15 is smaller than a curvature radius r2 in the area BA2. Further, the curvature radius r2 in the area BA2 is smaller than a curvature radius r3 in the area BA3.

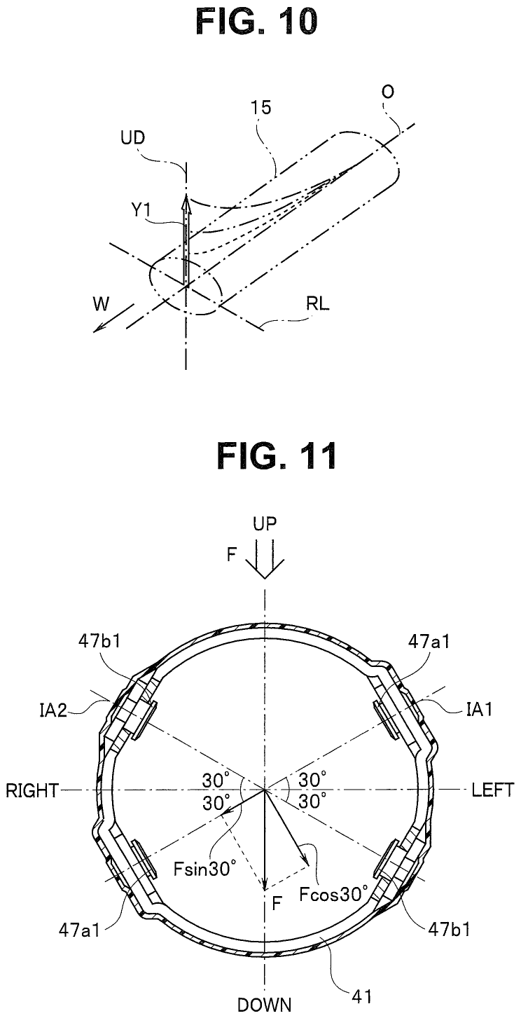

FIG. 10 is a diagram describing a bendable range in the up-down direction (UD direction) of the passive bending portion 15. FIGS. 11 to 13 are diagrams showing a component force applied to the rotation axis when pressing force of a large intestine is applied from the up direction to the passive bending portion 15 according to the present embodiment.

As shown in FIG. 10, the passive bending portion 15 is bendable by 360° around the central axis O toward the insertion direction W. However, as described above, the plurality of bending pieces 41 are coupled and included in the passive bending portion 15, and therefore maximum bending angles in the three areas BA1 to BA3 in the UD direction are not the same. In FIG. 10, a chain line shows the maximum bending angle in a part of the area BA1, a chain double-dashed line shows the maximum bending angle in a part of the area BA2, and a dotted line shows the maximum bending angle in a part of the area BA3.

FIG. 11 is a diagram showing a component force applied to the rotation axis when the pressing force of the large intestine is applied from the up direction to the area BA1 of the passive bending portion 15 according to the present embodiment.

As shown in FIG. 11, when an external force F is applied from the up direction (similar to an external force from the down direction) of the area BA1, a component force of F cos 30° acts on the third rotation axis IA1 in the bending of the passive bending portion 15. Since a component force of F sin 30° acts on the same axis as the third rotation axis IA1, the component force is cancelled and does not exert an influence on the bending of the passive bending portion 15. Although description is omitted in FIG. 11, the same component force also acts on the fourth rotation axis IA2.

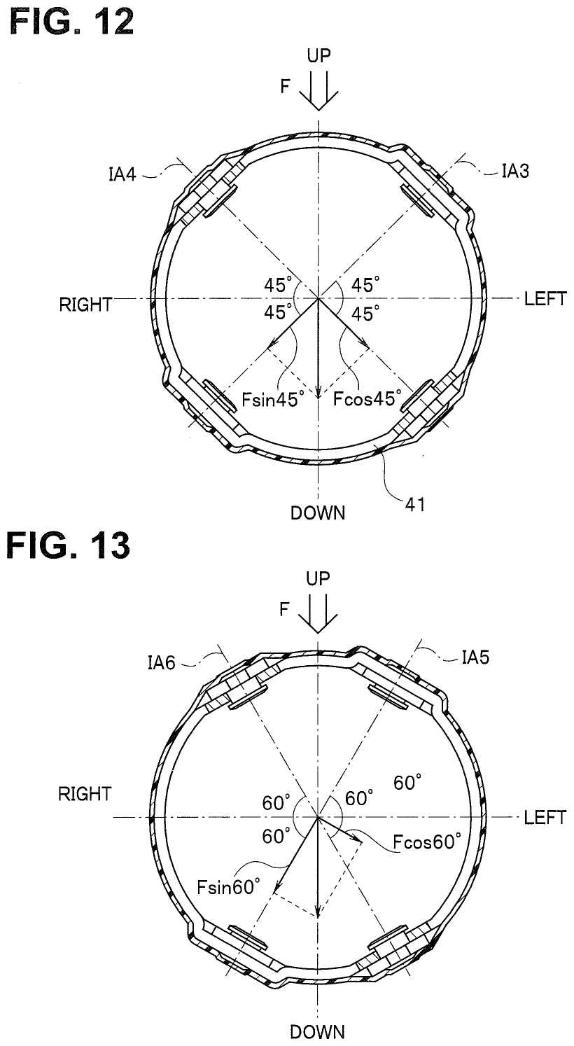

FIG. 12 is a diagram showing a component force applied to the rotation axis when the pressing force of the large intestine is applied from the up direction to the area BA2 of the passive bending portion 15 according to the present embodiment.

As shown in FIG. 12, when an external force F is applied from the up direction (similar to an external force from the down direction) of the area BA2, a component force of F cos 45° acts on the fifth rotation axis IA3 in the bending of the passive bending portion 15. Since a component force of F sin 45° acts on the same axis as the fifth rotation axis IA3, the component force is cancelled and does not exert an influence on the bending of the passive bending portion 15. Although description is omitted in FIG. 12, the same component force also acts on the sixth rotation axis IA4.

FIG. 13 is a diagram showing a component force applied to the rotation axis when the pressing force of the large intestine is applied from the up direction to the area BA3 of the passive bending portion 15 according to the present embodiment.

As shown in FIG. 13, when an external force F is applied from the up direction (similar to an external force from the down direction) of the area BA3, a component force of F cos 60° acts on the seventh rotation axis IA5 in the bending of the passive bending portion 15. Since a component force of F sin 60° acts on the same axis as the seventh rotation axis IA5, the component force is cancelled and does not exert an influence on the bending of the passive bending portion 15. Although description is omitted in FIG. 13, the same component force also acts on the eighth rotation axis IA6.

Accordingly, component forces applied to each of the rotation axes IA1 and IA2 when receiving the external force F applied to the up-down direction in the area BA1 are larger than component forces applied to each of the rotation axes IA3 and IA4 when receiving the external force F applied to the up-down direction in the area BA2, and therefore the area BA1 is easier to be bent than the area BA2.

Further, component forces applied to each of the rotation axes IA3 and IA4 when receiving the external force F applied to the up-down direction in the area BA2 are larger than component forces applied to each of the rotation axes IA5 and IA6 when receiving the external force F applied to the up-down direction in the area BA3, and therefore the area BA2 is easier to be bent than the area BA3.

Note that, as described above, a magnitude of a component force applied to each of the rotation axes differs according to the external force F applied to the up-down direction. Therefore, a thickness of a thinned portion of each of the bending pieces 41 may be partially changed according to a component force in an area.

As described above, the passive bending portion 15 provided on the proximal end side of the active bending portion 14 has two first bending pieces 41a and 41b that are rotatable around two first rotation axes IA1 and IA2 crossing at first angles θ1 and θ2 in a direction opposite to each other around the central axis of the insertion portion 2 with respect to the second direction (RL direction) orthogonal to the first direction (UD direction) when viewing the insertion portion 2 from the distal end side and two second bending pieces 41c and 41d that are rotatable around two second rotation axes IA3 and IA4 crossing at second angles θ3 and 04 different from the first angle in a direction opposite to each other around the central axis of the insertion portion 2 with respect to the second direction (RL direction). Further, the two second bending pieces 41c and 41d are provided on the proximal end side of the two first bending pieces 41a and 41b and absolute values of the second angles θ3 and 04 are larger than absolute values of the first angles θ1 and 02.

The bending piece 41b on the proximal end side of the two first bending pieces 41a and 41b is connected, by the two rivets 47a2, to the bending piece 41c on the distal end side of the two second bending pieces 41c and 41d.

The bending piece 41e is rotatable around the seventh rotation axis IA5 crossing at the fifth angle θ5 around the central axis of the insertion portion 2 with respect to the second direction (RL direction). Further, the pipe sleeve 46 is rotatable around the eighth rotation axis IA6 crossing at the sixth angle θ6 around the central axis of the insertion portion 2. A direction around the central axis at the first to sixth angles is set so as to be alternated in the longitudinal axis direction of the insertion portion 2.

Further, each of the bending pieces 41 of the passive bending portion 15 is rotatable around the corresponding rotation axis. Further, absolute values of the angles θ1 to θ6 with respect to the RL direction of the six rotation axes IA1 to IA6 are large from the distal end side to the proximal end side of the passive bending portion 15. The absolute values of the respective angles from the angles θ1 to θ6 are larger than 0° and are equal to or smaller than 60°.

### (Action)

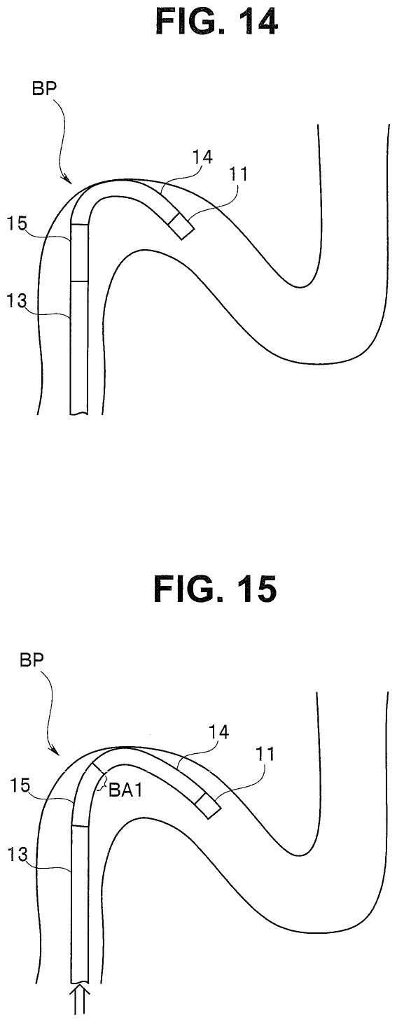

Operations in which the insertion portion 2 having the above-described configuration is operated and the insertion portion 2 is caused to pass through a flexed portion in the subject will be described. Here, an operation when the insertion portion 2 is inserted into the flexed portion in the large intestine will be described. FIGS. 14 to 17 are diagrams describing an operation in which the insertion portion 2 of the endoscope 1 according to the present embodiment is inserted into the flexed portion BP in the large intestine.

As shown in FIG. 14, when the insertion portion 2 is inserted into a flexed direction of the flexed portion BP, the operator presses the insertion portion 2 while bending the active bending portion 14 so that the distal end portion 11 is directed to the flexed direction of the flexed portion BP.

The operator operates the up-down bending operation knob 3a while viewing an endoscopic image displayed on a monitor. Further, the operator presses the insertion portion 2 while the active bending portion 14 is bent in any direction of the UD directions.

As shown in FIG. 15, when the insertion portion 2 is pressed, the active bending portion 14 is pressed against an intestinal wall, and therefore the passive bending portion 15 is also bent by moment of force received from the flexed portion such as an intestinal canal. However, at this time, as shown in FIG. 15, in the passive bending portion 15, the area BA1 in a distal end side part is easier to be bent, only in the UD direction, than the area BA2 in a central part and the area BA3 in a proximal end side part. Therefore, the distal end side part of the passive bending portion 15 is bent in any direction (namely, the flexed direction of the flexed portion BP) of the UD directions accorded with the flexed direction of the flexed portion.

As a result, when the insertion portion 2 is pressed, the area BA1 that is the distal end side part of the passive bending portion 15 is bent earlier than the areas BA2 and BA3. Therefore, it is difficult to generate a large force for knocking up the intestinal wall by the insertion portion 2.

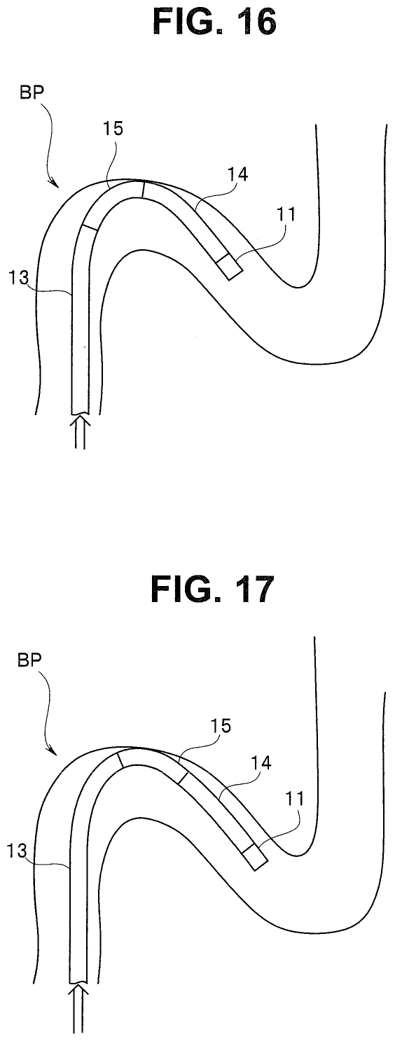

The distal end side part of the passive bending portion 15 is bent in the flexed direction (here, any direction of the UD directions) and performs an orientation of the bending direction of the passive bending portion 15. Therefore, as shown in FIG. 16, the area BA2 in the central part is also bent in the same flexed direction, and subsequently the area BA3 in the proximal end side part is also bent in the flexed direction (here, any direction of the UD directions) following the central part. As a result, as shown in FIG. 17, the passive bending portion 15 passes through the flexed portion BP.

As described above, the distal end side part of the passive bending portion 15 is easier to be bent, only in the UD direction, than the central part and the proximal end side part. Accordingly, while the active bending portion 14 is bent in a predetermined direction (here, any direction of the UD directions) in conformity to the flexed direction of the flexed portion BP, when the operator presses the insertion portion 2, the passive bending portion 15 smoothly enter the flexed direction of the flexed portion BP.

A conventional passive bending portion has a configuration in which the distal end side part is smaller than the proximal end side part in the curvature radius. However, when the distal end side part is not bent in the flexed direction of the flexed portion BP, the insertion portion 2 gets into a state of so-called stick phenomenon. Therefore, the operator cannot smoothly insert the insertion portion 2 in the flexed direction of the flexed portion BP and it may be necessary to reinsert the insertion portion 2.

However, according to the above-described embodiment, when the operator conforms the flexed direction of the flexed portion BP to a predetermined direction (here, any direction of the UD directions), bends the active bending portion 14, and presses the insertion portion 2 into the flexed portion BP, the distal end side part of the passive bending portion 15 is easy to be bent in the predetermined direction. Therefore, the passive bending portion 15 can be naturally bent in the flexed direction of the flexed portion BP from the distal end side part and advance along the flexed direction of the flexed portion BP.

As described above, according to the above-described embodiment, the insertion device in which insertability into the predetermined direction of the bending portion of the insertion portion is improved can be provided.

### Second Embodiment

According to the first embodiment, the passive bending portion 15 is configured so that the distal end side part of the passive bending portion 15 is easier to be bent in the predetermined direction (the up-down direction according to the first embodiment) and ease of bending in the predetermined direction is reduced toward the proximal end side of the passive bending portion 15. On the other hand, according to a second embodiment, the passive bending portion is configured so that a bending piece on the most distal end of the passive bending portion is bent only in a predetermined direction.

A configuration of an endoscope according to the present embodiment is substantially the same as the configuration of the endoscope 1 according to the first embodiment. Therefore, the same components as the components of the endoscope 1 according to the first embodiment will be denoted by the same reference numerals, and descriptions will be omitted. Further, only components different from the components of the endoscope 1 according to the first embodiment will be described.

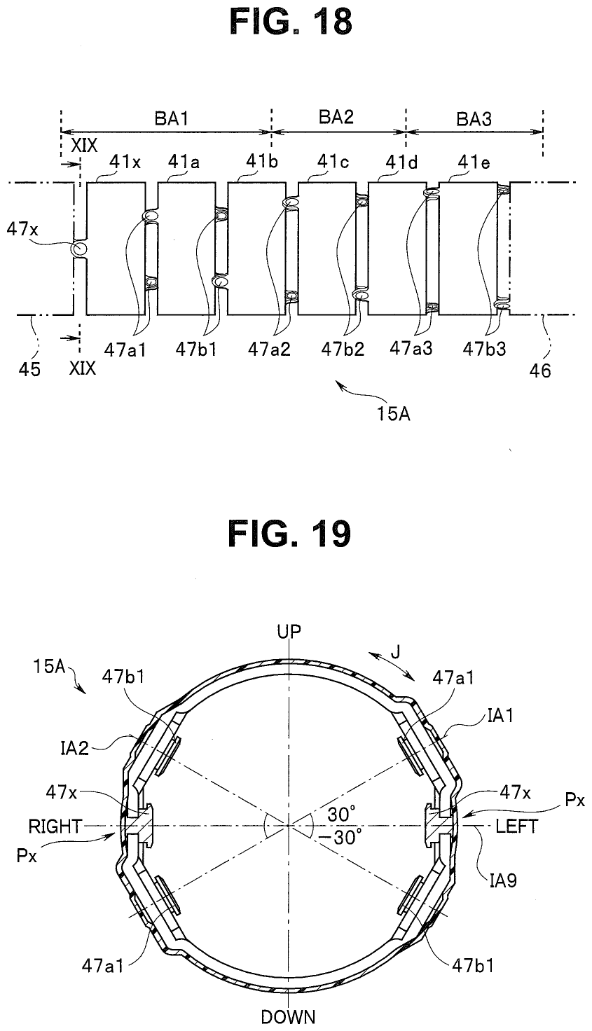

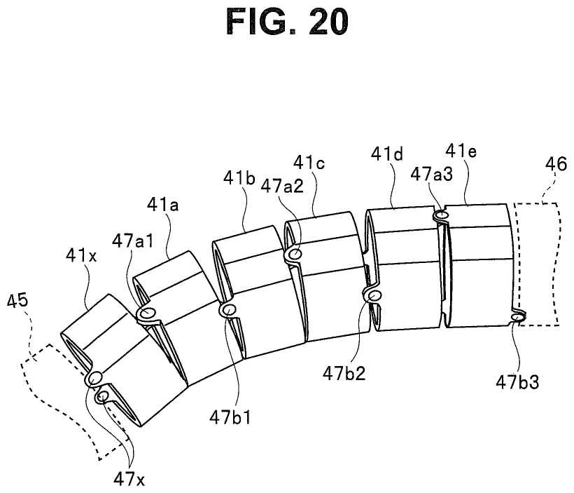

FIG. 18 is a front view of the plurality of bending pieces 41 showing a coupling relationship among the bending pieces 41 of a passive bending portion 15A according to the second embodiment. FIG. 18 shows only the plurality of bending pieces 41. FIG. 19 is a cross-sectional view along a XIX-XIX line shown in FIG. 18. FIG. 20 is a perspective view of the plurality of bending pieces of the bent passive bending portion 15A.

In FIG. 18, a coupling relationship among the bending pieces 41a to 41e is the same as the coupling relationship of the passive bending portion 15 according to the first embodiment.

According to the first embodiment, when the insertion portion 2 is viewed from the distal end side, the bending piece 41a and the pipe sleeve 45 are connected by the two rivets 47a1 in the two positions P1 on the third rotation axis IA1 inclined by the predetermined first angle θ1 (here, 30°) with respect to the first rotation axis RL.

However, according to the present embodiment, as shown in FIG. 19, when the insertion portion 2 is viewed from the distal end side, the bending piece 41a and a bending piece 41x at the most distal end are connected by the two rivets 47a1 in the two positions P1 on the third rotation axis IA1 inclined by the predetermined first angle θ1 (here, 30°) with respect to the first rotation axis RL. Further, when the insertion portion 2 is viewed from the distal end side, the bending piece 41x at the most distal end is coupled to the pipe sleeve 45 by two rivets 47x in two positions Px on the first rotation axis RL.

In other words, the passive bending portion 15A has the bending piece 41x provided on the distal end side of the two first bending pieces 41a and 41b. Further, when the insertion portion 2 is viewed from the distal end side, the bending piece 41x is rotatable around a rotation axis IA9 parallel to the RL direction.

As a result, the bending piece 41x at the most distal end of the passive bending portion 15A is rotatable only around the first rotation axis RL with respect to the pipe sleeve 45. Accordingly, the bending piece 41x at the most distal end of the passive bending portion 15A is bent only in the up-down direction. Therefore, when the operator causes the insertion portion 2 to pass through the flexed portion such as an intestinal canal, the operator conforms either direction of the up and down directions of the passive bending portion 15A to the flexed direction of the flexed portion and presses the insertion portion 2. In the case, since the bending piece 41x at the most distal end is bent only in the flexed direction, the plurality of bending pieces 41 coupled to the bending piece 41x is also bent in the same direction (namely, the flexed direction) as the direction of the bending piece 41x. In other words, the bending piece 41x at the most distal end functions so as to perform an orientation to the UD direction from the bending direction of the passive bending portion 15A.

Here, the bending piece 41x at the most distal end is one; further, one or more bending pieces 41x the same as the bending piece 41x may be coupled to the proximal end side of the bending piece 41x at the mot distal end. In other words, one or more bending pieces 41x coupled to the bending piece 41x at the most distal end by the two rivets 47x may be provided in the two positions Px on the first rotation axis RL.

As described above, when the insertion portion 2 is viewed from the distal end side, the bending piece 41x at the most distal end is connected to the pipe sleeve 45 of the active bending portion 14 in the two positions Px on the first rotation axis RL. Thereby, the bending direction of the passive bending portion 15A is oriented to the UD direction, and thereby the insertability is improved into the predetermined direction of the passive bending portion 15A.

Therefore, according to the above-described present embodiment, it is possible to provide the insertion device in which the insertability into the predetermined direction of the bending portion of the insertion portion 2 is improved.

### Third Embodiment

According to the second embodiment, when the insertion portion 2 is viewed from the distal end side, the bending piece 41x at the most distal end is connected to the pipe sleeve 45 of the active bending portion 14 in the two positions Px on the first rotation axis RL. On the other hand, according to a third embodiment, when the insertion portion 2 is viewed from the distal end side, at least one bending piece 41 coupled to the bending piece 41 on the distal end side by two rivets is provided among the plurality of bending pieces 41 in two positions on the first rotation axis RL in the area BA1 on the distal end side.

A configuration of an endoscope according to the present embodiment is substantially the same as the configuration of the endoscope 1 according to the first and second embodiments. Therefore, the same components as the components of the endoscope 1 according to the first and second embodiments will be denoted by the same reference numerals, and descriptions will be omitted. Further, only components different from the components of the endoscope 1 according to the first and second embodiments will be described.

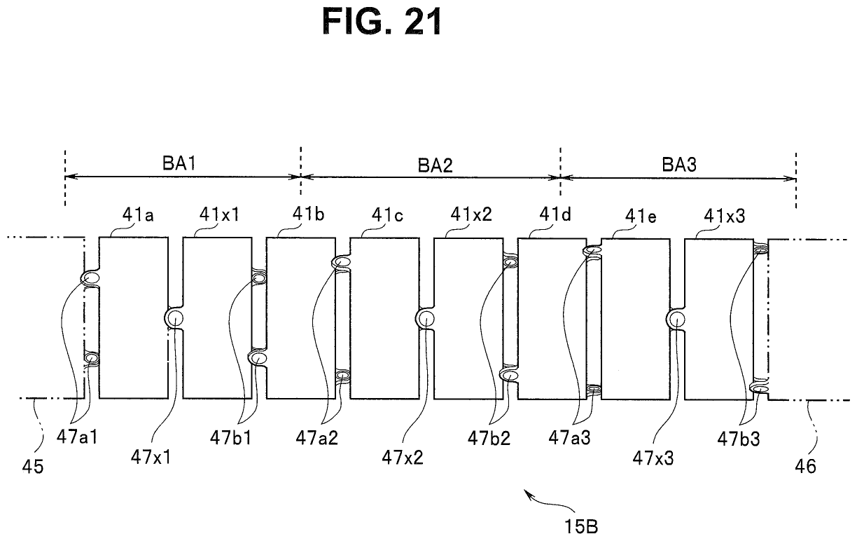

FIG. 21 is a front view of the plurality of bending pieces 41 showing a coupling relationship among the bending pieces 41 of a passive bending portion 15B according to the third embodiment.

As shown in FIG. 21, the bending piece 41x1 is provided between two bending pieces 41a and 41b in the area BA1 on the distal end side of the passive bending portion 15B.

More specifically, a bending piece 41x1 is connected to the bending piece 41a by two rivets 47x1 in the two positions Px on the first rotation axis RL. The bending piece 41x1 and the bending piece 41b are connected by two rivets 47b1 in two positions P2 on the fourth rotation axis IA2 inclined by the predetermined second angle θ2 (here, −30°) with respect to the first rotation axis RL.

In other words, the passive bending portion 15B has the bending piece 41x1 provided between the two first bending pieces 41a and 41b. Further, when the insertion portion 2 is viewed from the distal end side, the bending piece 41x1 is rotatable around the rotation axis IA9 parallel to the RL direction.

As a result, the bending piece 41x1 in the area BA1 on the distal end side of the passive bending portion 15B is connected to the bending pieces 41a and 41b in the two positions Px on the first rotation axis RL. Therefore, an orientation to the bending direction is performed in the area BA1 of the passive bending portion 15B, and thereby the insertability into the predetermined direction of the passive bending portion 15B is improved.

Further, as shown in FIG. 21, a bending piece 41x2 is provided also between two bending pieces 41c and 41d in the area BA2 in the middle of the passive bending portion 15B. In other words, the bending piece 41x2 is provided between the two bending pieces 41c and 41d and, when the insertion portion 2 is viewed from the distal end side, the bending piece 41x2 is rotatable around the rotation axis IA9 parallel to the RL direction. Further, the bending piece 41x3 is provided also between the pipe sleeve 46 and the bending piece 41e in the area BA3 on the proximal end side of the passive bending portion 15B. The bending piece 41x3 is also rotatable around the rotation axis IA9 parallel to the RL direction when the insertion portion 2 is viewed from the distal end side.

Therefore, according to the above-described present embodiment, it is possible to provide the insertion device in which the insertability into the predetermined direction of the bending portion of the insertion portion 2 is improved.

Note that, according to the third embodiment, the three bending pieces 41x1, 41x2, and 41x3 are provided in the areas BA1, BA2, and BA3. However, one or more bending pieces 41x1 just may be provided in the area BA1.

The bending pieces 41x2 and 41x3 that rotate around the rotation axis IA9 are provided also in the areas BA2 and BA3. Further, an orientation to the UD direction is performed also in the middle of and on the proximal end side of the passive bending portion 15B, and thereby the insertability into the predetermined direction of the passive bending portion 15B is improved. Further, the bending piece 41x1 is provided only in the area BA1, and thereby an orientation may be performed only on the distal end side of the passive bending portion 15B.

The distal end side part of the passive bending portion 15B is made easier to be bent, in the predetermined direction, than the proximal end side part of the passive bending portion 15B, and thereby when the operator presses the insertion portion 2, a passing property at the flexed portion BP is excellent.

Note that the bending piece 41x at the most distal end according to the second embodiment may be further provided at the distal end portion of the passive bending portion 15B according to the third embodiment.

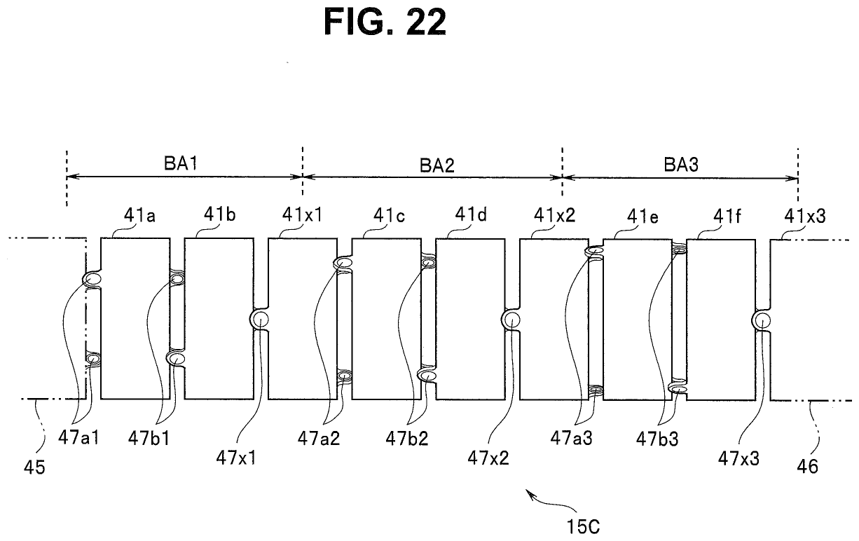

Further, according to another embodiment of the third embodiment, the passive bending portion 15B may have a configuration as shown in FIG. 22. FIG. 22 is a front view of the plurality of bending pieces 41 showing a coupling relationship among the bending pieces 41 of the passive bending portion 15B according to a modification of the third embodiment. As shown in FIG. 22, from the distal end side of the passive bending portion 15B, the plurality of bending pieces 41 may be provided in the order of the bending piece 41a constituting the area BA1, the bending piece 41b and the bending piece 41x1 constituting the area BA1, the bending piece 41c constituting the area BA2, the bending piece 41d and the bending piece 41x2 constituting the area BA2, the bending piece 41e constituting the area BA3, and the bending piece 41f and the bending piece 41x3 constituting the area BA3, and the like. In FIG. 22, the pipe sleeve 46 is shown as the bending piece 41x3.

In other words, in the plurality of bending pieces 41, the bending piece on the proximal end side of the two first bending pieces 41 is a bending piece that is different from the bending piece 41 on the distal end side of the two second bending pieces 41.

Even in the embodiment, similarly to the third embodiment, the insertability into the predetermined direction of the passive bending portion 15 is improved.

As described above, according to each of the above-described embodiments, it is possible to provide the insertion device in which the insertability into the predetermined direction of the bending portion of the insertion portion is improved.

The present invention is not limited to the embodiments described above, and various changes and modifications may be made without departing from the gist and scope of the present invention.

What is claimed is:

1. An insertion device comprising:
an insertion portion that is inserted into a subject from a distal end side in a longitudinal axis direction, wherein
the insertion portion includes:
a distal end portion that is provided at a distal end of the insertion portion;
a first bending portion that is provided on a proximal end side of the distal end portion and is configured to be bent in a first direction according to a bending operation of an operator;
a second bending portion that is provided on a proximal end side of the first bending portion and is passively bent by receiving an external force without being bent by the bending operation of the operator; and
a flexible tube that is provided on a proximal end side of the second bending portion and has flexibility,
the second bending portion includes:
a first area including a first coupling portion that couples two adjacent bending pieces rotatably around a first rotation axis crossing in a positive direction at a first angle around a central axis of the insertion portion with respect to a second direction orthogonal to the first direction and a second coupling portion that couples the two adjacent bending pieces rotatably around a second rotation axis crossing in a negative direction at the first angle around the central axis with respect to the second direction; and
a second area including a third coupling portion that is provided on a proximal end side from the first area and couples another two adjacent bending pieces rotatably around a third rotation axis crossing in the positive direction at a second angle around the central axis with respect to the second direction and a fourth coupling portion that couples the other two adjacent bending pieces rotatably around a fourth rotation axis crossing in the negative direction at the second angle around the central axis with respect to the second direction, and
an absolute value of the second angle is larger than an absolute value of the first angle.

2. The insertion device according to claim 1, wherein
the first coupling portion through the fourth coupling portion are arranged so that a direction around the central axis with respect to the second direction is alternated in the longitudinal axis direction.

3. The insertion device according to claim 1, wherein
the first area and the second area are adjacent to each other.

4. The insertion device according to claim 1, wherein
a bending piece at a most proximal end in the first area is a bending piece at a most distal end in the second area.

5. The insertion device according to claim 4, wherein
the bending piece at the most proximal end in the first area includes the second coupling portion that is rotatable around the second rotation axis on the distal end side and includes the third coupling portion that is rotatable around the third rotation axis on the proximal end side.

6. The insertion device according to claim 1, wherein
the first area includes:
a first bending piece including the first coupling portion that is rotatable around the first rotation axis on the distal end side; and
a second bending piece that is coupled rotatably around the second rotation axis on a proximal end side of the first bending piece.

7. The insertion device according to claim 1, wherein
a first bending piece provided at a most distal end in the first area is a pipe sleeve that connects the first bending portion and the second bending portion.

8. The insertion device according to claim 1, wherein
a first bending piece provided at a most distal end in the first area includes, on the distal end side, a fifth coupling portion that is rotatable around a rotation axis parallel to the second direction when the insertion portion is viewed from the distal end side.

9. The insertion device according to claim 1, wherein
the first area includes:
a first bending piece including the first coupling portion that is rotatable around the first rotation axis on the distal end side;
a second bending piece that is coupled rotatably around a rotation axis parallel to the second direction on a proximal end side of the first bending piece; and
a third bending piece that is coupled rotatably around the second rotation axis on a proximal end side of the second bending piece.

10. The insertion device according to claim 9, wherein
the second area includes:
a fourth bending piece including the third coupling portion that is rotatable around the third rotation axis on the distal end side;
a fifth bending piece that is coupled rotatably around a rotation axis parallel to the second direction on a proximal end side of the fourth bending piece; and
a sixth bending piece that is coupled rotatably around the fourth rotation axis on a proximal end side of the fifth bending piece.

11. The insertion device according to claim 1, wherein
absolute values of the first angle and the second angle are larger than 0° and are equal to or smaller than 60°.

12. The insertion device according to claim 1, wherein
the first bending portion includes a plurality of serially coupled bending pieces.

13. The insertion device according to claim 1, wherein
the first bending portion is bendable also in the second direction according to the bending operation of the operator when the insertion portion is viewed from the distal end side.

14. The insertion device according to claim 1, wherein
the distal end portion includes an image acquisition apparatus that picks up an image of the subject, and
the first direction is substantially parallel to an up-down direction of an image obtained by the image acquisition apparatus and displayed on a screen of a display apparatus and the second direction is substantially parallel to a left-right direction of the image.
